(19) United States
(12) Patent Application Publication
(10) Pub. No.: US 2015/0045863 A1
(43) Pub. Date: Feb. 12, 2015

(54) EXPANDABLE ELECTRODES AND METHODS FOR TREATING TISSUES

(71) Applicant: Boston Scientific Scimed, Inc., Maple Grove, MN (US)

(72) Inventors: Eric K. LITSCHER, Hopkinton, MA (US); Jason WEINER, Grafton, MA (US); Hol YOU, Lowell, MA (US)

(73) Assignee: Boston Scientific Scimed, Inc.

(21) Appl. No.: 14/452,338

(22) Filed: Aug. 5, 2014

Related U.S. Application Data
(60) Provisional application No. 61/863,143, filed on Aug. 7, 2013.

(51) Int. Cl. A61N 1/05

(52) U.S. Cl. CPC A61N 1/05
USPC 607/116; 29/825

(57) ABSTRACT

Provided are devices for treating tissue. In particular, the devices may include a first conductive elongate member having a first distal bend portion, a first leg portion extending proximally from the first distal bend portion, and a second leg portion extending proximally from the first distal bend portion. The device may also include a second conductive elongate member having a second distal bend portion, a third leg portion extending proximally from the second distal bend portion, and a fourth leg portion extending proximally from the second distal bend portion. The second distal bend portion may be disposed proximally of the first distal bend portion. The device may also include an elongate pull member affixed to the second distal bend portion.

### CROSS-REFERENCE TO RELATED APPLICATIONS

This application claims the benefit of priority from U.S. Provisional Application No. 61/863,143, filed on Aug. 7, 2013, the entirety of which is incorporated by reference herein.

### FIELD

Embodiments of the disclosure relate generally to medical devices and methods of treating medical conditions. In particular, embodiments of the present disclosure relate to medical devices for treating internal walls of lumens of the human body, for example, treating airway walls to effect lung disease and more particularly to medical devices which transfer energy to airway tissue.

### BACKGROUND

Obstructive pulmonary disease includes chronic obstructive pulmonary disease (COPD) and reversible obstructive disease (e.g. asthma, and some reversible aspects of COPD). Examples of COPD include emphysema, and chronic bronchitis. The reversible aspects of COPD generally describe excessive mucus production in the bronchial tree. Usually, there is a general increase in bulk (hypertrophy) of the large bronchi and chronic inflammatory changes in the small airways. Excessive amounts of mucus are found in the airways and semisolid plugs of mucus may occlude some small bronchi. Also, the small airways are narrowed and show inflammatory changes. The reversible aspects of COPD include partial airway occlusion by excess secretions, and airway narrowing secondary to smooth muscle contraction, bronchial wall edema and inflation of the airways. Asthma is a inflammatory disease of the airways, the symptoms of which include bronchoconstriction, excessive mucus production, and inflammation and swelling of airways. These symptoms restrict airflow, making it difficult for asthma sufferers to breathe, and may cause shortness of breath (dyspnea), wheezing, chest tightness, and coughing. Asthma is also characterized by additional acute airway narrowing via contraction of hyper-responsive airway smooth muscle due to one or more stimuli. Such stimuli may be allergenic or non-allergenic. Examples of allergenic stimuli include smoke, pollen, pet dander, dust mites, bacterial or viral infections, mold, dust, and airborne pollutants. Non-allergenic stimuli may include, but are not limited to, exercise, or exposure to cold, dry air.

The airway wall of asthma sufferers may be remodeled due to the chronic nature of the disease. These structural changes, such as thickening or edema, of the airway wall may further affect the function of the airway wall and influence airway hyper responsiveness. Other physiological changes associated with asthma include mucus plugging due to excessive mucus production, as well as ongoing epithelial denudation and repair. Epithelial denudation may expose the underlying tissue to substances that may not normally come in contact with the tissue, further reinforcing the cycle of cellular damage and inflammatory response.

Currently, asthma is managed by a combination of stimulus avoidance and pharmacolostimulus avoidance, which is accomplished via systematic identification, and minimization of contact with each type of stimuli. It may, however, be impractical to avoid all potential stimuli. Asthma is also often managed pharmacologically. Anti-inflammatories, such as corticosteroids, and long-term bronchodilators are often used for long-term control, whereas acute exacerbations are often managed through use of short-acting bronchodilators. Both long-term and short-term pharmological treatments may be repeated with regular use of the prescribed drugs. However, high doses of corticosteroid anti-inflammatory drugs can have serious side effects that require careful management, and some patients may be resistant to steroid treatment. In addition, patient compliance with pharmacologic management and stimulus avoidance is often a barrier to successful asthma management.

Accordingly, a need exists for asthma treatment, which improves airflow without the need for patient compliance and the potential harmful side effects of drugs.

### SUMMARY

Embodiments of the present disclosure relate to devices for treating tissue, such as in organs with lumens having an inner wall (e.g. to access anatomy, nerve, muscle or tissue wall).

In accordance with an aspect of the present disclosure, a device for treating tissue may include a first conductive elongate member having a first distal bend portion, a first leg portion extending proximally from the first distal bend portion, and a second leg portion extending proximally from the first distal bend portion. The device may also include a second conductive elongate member having a second distal bend portion, a third leg portion extending proximally from the second distal bend portion, and a fourth leg portion extending proximally from the second distal bend portion. The second distal bend portion may be disposed proximally of the first distal bend portion. The device may also include an elongate pull member affixed to the second distal bend portion.

Various embodiments of the device may include one or more of the following features: the first and second distal bend portions may not be in the same plane, each of the first, second, third, and fourth leg portions may form a cage configuration adapted to transition from a collapsed state to an expanded state, the first and second distal bend portions may not be directly attached to each other. The device may further include an alignment portion disposed on the pull wire and configured to align the first and second leg portions of the first conductive elongate member and the third and fourth leg portions of the second conductive elongate member at predetermined relative orientations, the device also may include a proximal retainer disposed over a proximal portion of the elongate pull member, the proximal retainer may comprise an internal lumen having a cross-section configured to retain proximal portions of the first and second conductive elongate members and a proximal portion of the alignment portion, the internal lumen of the proximal retainer may comprise a stop configured to abut proximal ends of the first and second conductive elongate members. The device may also include thermocouple wires attached to one or more portions of the first and second conductive elongate members, a distal retainer disposed over the first and second distal bend portions, the distal retainer may be directly and fixedly attached to the second conductive elongate member, and the distal retainer may be conductive and may not be directly fixedly attached to the first conductive elongate member.

In accordance with another aspect, a device for treating tissue that may include a first conductive elongate member having a first distal bend portion positioned in a first plane, a first leg portion extending proximally from the first distal bend portion, and a second leg portion extending proximally from the first distal bend portion. The device may include a second conductive elongate member having a second distal bend portion positioned in a second plane different from the first plane, a third leg portion extending proximally from the second distal bend portion, and a fourth leg portion extending proximally from the second distal bend portion. The second distal bend portion may be disposed proximally of the first distal bend portion, and/or an elongate pull member may be directly and fixedly attached to an inner surface of the second distal bend portion. The device may also include an alignment element disposed on the pull member and configured to align the first and second leg portions of the first conductive elongate member and the third and fourth leg portions of the second conductive elongate member at predetermined relative orientations.

Various embodiments of device may include one or more of the following features: a distal end of the elongate pull member having a diameter greater than a diameter of a proximal portion of the elongate pull member may be directly and fixedly attached to the inner surface second distal bend portion, each of the first, second, third, and fourth leg portions may form a cage configuration adapted to transition from a collapsed state to an expanded state, the first and second distal bend portions may not be directly attached to each other, a proximal retainer may be disposed over a proximal portion of the elongate pull member, the proximal retainer may comprise an internal lumen having a cross-section configured to retain proximal portions of the first and second conductive elongate members and a proximal portion of the alignment element, the lumen of the proximal retainer may include a stop configured to abut proximal ends of the first and second conductive elongate members. The device also may include thermocouple wires attached to one or more portions of the first and second conductive elongate members, and a distal retainer disposed over the first and second distal bend portions, the distal retainer may be directly and fixedly attached to the second conductive elongate member and not directly fixedly attached to the first conductive elongate member.

Another aspect of the present disclosure includes a method of manufacturing a device for treating tissue, and may include steps of bending a first elongate component to form a first set of two leg portions extending proximally of a first bent portion, bending a second elongate member to form a second set of two leg portions extending proximally of a second bent portion, and positioning the second bent portion approximately perpendicular to and proximal of the first bent portion.

Various embodiments of the method may include one or more of the following features: attaching a distal end of a pull wire to a proximal side of the second bent portion, disposing a retainer over the first and second bent portions, and directly fixing the retainer to only one of the first and second bend portions.

Additional objects and advantages of the present disclosure will be set forth in part in the description which follows, and in part will be understood from the description, or may be learned by practice of the claimed invention. The objects and advantages of the claimed invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIGS. 1A and 1B are exploded partial perspective views of an expanded device, according to an embodiment of the present disclosure.

FIGS. 2A, 2B, and 2C are partial perspective views of components of a device, according to an embodiment of the present disclosure.

FIG. 3 is a partial perspective view of a pull elongate component of the device, according to an embodiment of the present disclosure.

FIG. 4 is a cross-sectional view of an exemplary distal end of a device, according to an embodiment of the present disclosure.

FIG. 5 is a perspective view showing retainer and delivery tube components, according to an embodiment of the present disclosure.

FIGS. 6A and 6B illustrate exemplary retainer components, according to embodiments of the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts. The term “distal” refers to the end farthest away from a medical professional when introducing a device into a patient. By contrast, “proximal” refers to the end closest to the medical professional when placing a device into the patient.

### Overview

Embodiments of the present disclosure relate to devices, systems, and methods for treating tissue, such as in organs with lumens having an inner wall (e.g. to access anatomy, nerve, muscle or tissue wall). In particular, embodiments of the present disclosure relate to devices, systems, and methods which may transfer energy to lung tissue in order to, among other things, reduce airway resistance, for example by reducing, shrinking, debulking, and/or eliminating airway tissue, including airway smooth muscle.

### Exemplary Embodiments

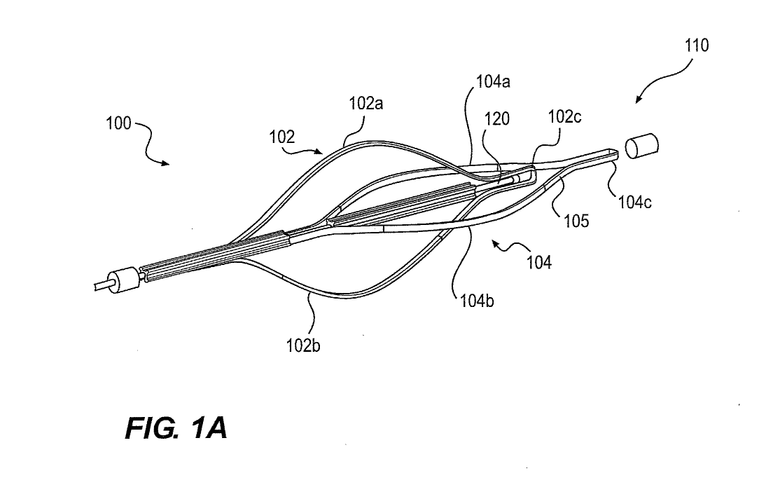

FIGS. 1A and 1B illustrate two exploded partial perspective views of an exemplary device 100 for transferring energy to airway tissue, according to embodiments of the present disclosure. Particularly, FIG. 1A illustrates a distal portion of device 100 prior to formation of a cage or basket configuration 101, and FIG. 1B illustrates a distal portion of device 100 after formation of the cage or basket configuration 101. The cage or basket configuration 101 may include multiple electrodes, e.g. two, three, four, five, or any other number of electrodes.

Each of the electrodes, for example, electrodes 102 and 104, may be oriented in different planes from each other, at any suitable non-parallel angular orientation. For example, each electrode may be oriented at 10°, 20°, 30°, 40°, 50°, 60°, 70°, 80°, 90° or any other suitable angle relative to each other. The electrodes 104 and 106 may be oriented at a uniform or non-uniform angle from an adjacent electrode. For example, a first electrode may be oriented 15° relative to a second electrode and 60° relative to a third electrode. In another example, a second and third electrode may be oriented 45° and 90° respectively, relative to a first electrode. As shown in FIGS. 1A and 1B, for instance, the cage 101 may have two electrodes—first electrode 102 and second electrode 104, which may be oriented in different planes, for example, at about 90° relative to each other.

The electrode cage 101 may have any suitable shape, size, and geometry for expanding in the airway. For example, the cage 101 may have a spherical, elliptical, tapered, tubular, rectangular, triangular, or any other suitable shape, or geometry.

The cage 101 may be transitioned from a first collapsed state to a second expanded state by transitioning one or more of its component electrodes 102 and 104 from a collapsed state to an expanded state. Expansion may be accomplished by any suitable means. In one embodiment, the electrodes may be manufactured using a material having a suitable width, length, thickness, and modulas such that when the distal tip of the cage 101 is pulled proximally, the electrodes 104 and 106 may buckle with minimal yield.

In another embodiment, the electrodes 102 and 104 may be manufactured using a self-expanding material. The self-expanding material may be manufactured using any suitable material for insertion in the airway that may be capable of self-expansion. Examples of such materials may include one or more metals, bimetallic metals, piezo electric, metal alloys, such as, nitinol, and/or polymer materials. For example, the self-expanding material may transition from the first, collapsed state to the second, expanded state when placed at the desired location.

In another embodiment, all or a portion of the electrodes 102 and 104 of the expandable cage 101 may be made of resilient material that may be held in a collapsed state by a holding component, such as a sheath or sleeve 124 that may be disposed over all or a portion of the expandable cage 101 to bias the cage 101 to the collapsed state. The holding component may be made of any suitable material and have any suitable shape and size, for holding the expandable cage 101 in a collapsed state. The holding component may be removed from the expandable cage 101, to allow the expandable cage 101 to transition from a collapsed state to an expanded state. For example, the holding component 124 may be removed by axially moving the holding component 124 from the collapsed cage 101, for example, moving the holding component 124 in a proximal direction.

In another example, the expandable cage 101 may transition from a collapsed state to an expanded state with the aid of an additional component. For instance, the expandable cage 101 may be delivered to the desired location in a collapsed state and then expanded by engaging a wire or any suitable external device (e.g. an inflatable member) to transition the expandable cage to an expanded state.

Each electrode 102 and 104 forming the cage configuration 101, may be formed from an elongate component, such as a metal wire. The elongate component may have any suitable size, shape, or geometry. For example, the cross-sectional shape of the elongate component may be flat, beveled, rounded, or any other suitable shape as desired. Each elongate component may have the same or different cross-sectional shapes and sizes and may have a variable cross-section along the length.

The elongate component may be formed to have multiple legs such as five or more, four, three, two or any suitable number of legs. For example, as shown in FIGS. 1A and 1B, each electrode 102 and 104 may be formed by any suitable manner to have multiple legs, such as electrode legs 102a, 102b, 104a, and 104b, respectively.

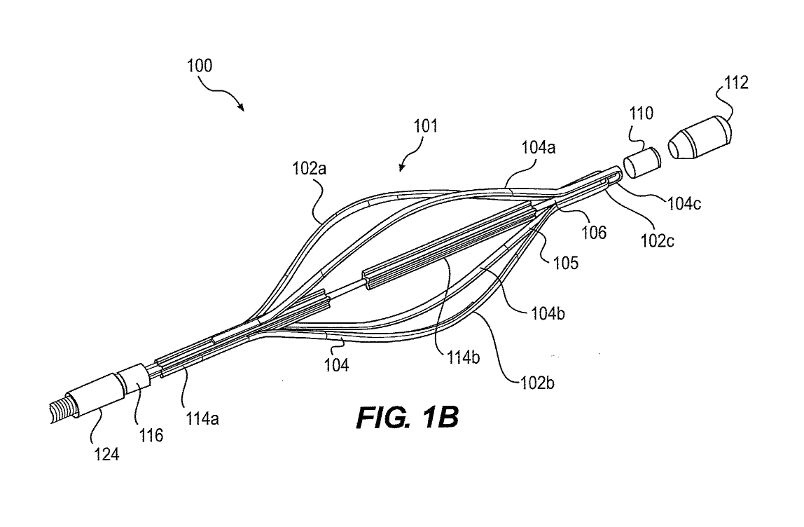

For example, the elongate component may be stamped and rolled or bent to form the electrode legs. As shown in FIG. 2A, the elongate component of electrode 102 may be bent to form one or more bends, such as bend 102c, from which two or more electrode legs 102a and 102b may extend. The bend(s) 102c may have any suitable size, shape, or geometry. As shown in FIGS. 2A, 2B, and 2C, for example, the bend portion 102c of the first electrode 102 may have a U-shape having a width configured so that a distal end 108 of a pull wire 106 may engage the inner surface of the bend portion 102c and transfer energy to the electrodes.

Each electrode leg extending from the bend may be spaced apart from the other electrode leg at a uniform or non-uniform distance and may form any suitable shape. For example, as shown in FIG. 2B, in an expanded state, the electrode legs 102a and 102b of first electrode 102 may extend from the bend portion 102c of the first electrode 102c to form an elliptical shape with proximal and distal tapered portions. One or more portions of each of the electrodes 102 and 104 may be strengthened or relaxed using any suitable method. For example, the bend portion 102c and 104c of the electrodes 102 and 104 may be annealed in order to remove internal stresses and strengthen that portion of the electrode 102 and 104 and allow for reduced bend radius. The tolerances of the bend portions 102c and 104c also may be configured to allow the electrode legs 102a, 102b, 104a, and 104b to fit in a retainer, such as a distal retainer 110.

Each electrode 102 and 104 may be manufactured using any material or combination of materials capable of being expanded (as discussed above) and capable of conducting energy. Each of the electrodes 102 and 104 also may be manufactured using the same or different materials. For example, each electrode may be manufactured using a work-hardened material, such as a metal, metal alloy, polymer, or other energy conducting materials. The electrodes 102 and 104 may have a high elastic modulus, such as an elastic modulus characteristic of stainless steel, or nitinol, so that the electrodes may be formed into a shape suitable for insertion in the body and or through a bronchoscope or other suitable delivery system. Examples of types of stainless steel that may be used in manufacturing the electrodes 102 and 104 may include, 304V stainless steel or any other suitable stainless steel.

The electrodes 102 and 104 formed from the respective elongate components may have any suitable size, shape, and geometry for insertion in the body and/or through a bronchoscope or other suitable delivery system, such size, shape, and geometry may be uniform or non-uniform along the length of the electrode. Each electrode 102 and 104 may have the same or different shapes, sizes, and/or geometries. For example, each electrode may have a rectangular shape. The electrodes 102 and 104 also may include one or more structural features along one or more portions, such as beveled portions, rounded portions, jagged portions, smooth portions, roughened portions, gaps, holes, ridges, tabs, slits, protrusions, or any other structural features.

For example, the electrodes 102 and 104 may include protrusions, which may be formed on the electrodes at any time prior to use, for example, prior to or after formation of the electrode legs 102 and 104, and/or prior to or after formation of the cage configuration 101. For example, the protrusions may include barbs, tines and/or other protrusions to allow temporary holding/re-positioning of the device 100 in the desired location as it is translated to the desired locations and delivery system.

The electrodes 102 and 104 may be RF energy based electrodes, which may be either bipolar or monopolar to transfer heat to the tissue. The frequency of the RF energy may be selected to be in any suitable range to heat tissue, for example, in the 400 kHz range or any other standard medical range used in electro-surgical applications.

In one embodiment, the electrodes 102 and 104 may have a monopolar configuration in which one of the electrodes may serve as an active electrode and another electrode may serve as a return electrode. The active electrode may be smaller than the return electrode. Therefore, the active electrode may have a high current density and relatively high tissue impedance, which may result in heating at the active electrode. The return electrode may have a lower current density and the same tissue impedance as the active electrode. Therefore, minimal heating may occur at the return electrode. According to the monopolar configuration, current may pass through the patients entire body and therefore, the exact position of the return electrode on the tissue may not be critical. The monopolar configuration also may require a single isolated electrode so that the entire cage 101 may include active electrode transmitting current. Therefore, the monopolar configuration may not require any isolation between adjacent electrodes and the electrodes 102 and 104 may be spaced diametrically equal to achieve even heating between electrodes 102 and 104.

According to another embodiment, the device 100 having the cage configuration 101 with electrodes 102 and 104 may be bipolar. In the bipolar configuration, both the active electrode and return electrode functions may be performed at the site of surgery. Each of the electrodes 102 and 104 may be isolated and the tissue between the electrodes may be included in the electrical circuit and therefore, no patient return electrode may be needed. For example, if the cage configuration 101 is bipolar cage, there may be an equal number of active electrodes and return electrodes spaced evenly around the cage to achieve consistent tissue heating. RF energy may be transferred to the electrodes 102 and 104 through the pull wire 106, catheter shaft, thermocouple wires 120, and/or any supplementary wires.

All or one or more portions of the electrodes 102 and 104 may include a coating, having, for example, lubricious, and/or therapeutic properties (e.g. antibiotic, anti-inflammatory, anesthetic), and may be porous, conductive, and/or insulating along all or a portion or portions of the length of the electrode. The coating may be applied to each of the electrodes at any time prior to use, for example, prior to or after formation of the electrode legs 102 and 104 and/or prior to after formation of the cage configuration 101.

In addition or alternatively, all or one or more portions of each of the electrodes 102 and 104 may have any suitable uniform or non-uniform surface features, such as a surface roughness, or pattern formed prior to bending and/or assembly of the cage configuration 101. For example, all or one or more portions or the electrodes may have one or more protrusions, bumps, indentations, grooves, ribs, etc., along the length of the electrode. Alternatively, the electrodes may have uniform smoothness.

Each of the electrodes may have insulated portions 118a and 118b, see FIG. 2A. For example, each of the electrode legs 102a, 102b, 104a, and 104b may have proximal end portions 118a and distal end portions 118b that may be covered with insulation. The insulation may be accomplished using any suitable means, such as via coating of the electrodes using vapor deposition, such as parylene vapor deposition, other thin layer coating methods, or any other suitable methods. The electrode legs 102a, 102b, 104a, and 104b may be insulated prior to bending and/or assembly of the cage configuration 101.

A portion of each of the electrode legs 102a, 102b, 104a, and 104b may be connected to thermocouple (TC) wires 120. The TC wires 120 may be connected to circuitry for monitoring parameters of energy transfer of, for example, voltage, current, power, impedance, as well as temperature. This information communicated via the TC wires 120 may be used by the power supply to control the amount of energy delivered to the electrodes. The TC wires 120 may be disposed on any portion or portions of the electrode legs 102a, 102b, 104a, and 104b, for example on an inside, outside, top or bottom surface. The TC wires 120 may be disposed on any portions of the electrode at any time prior to use, for example, prior to or after formation of the electrode legs 102 and 104 and/or prior to after formation of the cage configuration 101 by any suitable means. For example, the TC wires 120 may be soldered, welded, attached using adhesive or otherwise attached to electrodes. The TC wires 120 may be adapted to sense temperature of bodily fluid and/or tissue and may be connected at a proximal end, to an electrical connector.

The cage configuration 101 may be formed by placing the outer surface of a distal bend of one electrode at a position relative to the inner surface of a distal bend of another electrode. The distal bend of each electrode may be oriented at any suitable angle relative to the distal bend of the other electrodes. For example, the outer surface of a distal bend of one electrode may be disposed adjacent to the inner surface of a distal bend of another electrode and may not be in direct contact with each other. For example, as shown in in FIG. 1B, the outer surface of distal bend 102c may be positioned adjacent to and oriented about 90° relative to the inner surface of distal bend 104c having an insulating coating 118. Each electrode leg forming the electrode cage configuration may be spaced apart from an adjacent electrode leg at any suitable angle. For example, each electrode leg 102a, 102b, 104a, and 104b may be spaced at approximately 90° from an adjacent electrode leg and/or any even diametrical spacing of the electrodes. In a bipolar configuration, such even spacing may allow energy to pass from one electrode to an adjacent electrode.

The device 100 may include an energy transfer component adapted to transfer energy from a power source to the electrodes 102 and 104. The energy transfer component may be any shape, size, or geometry suitable for transferring energy. The energy transfer component may be manufactured using any suitable material for transferring energy from a power source to the electrodes 102 and 104, such a metal or any other material. For example, the energy transfer component may be made of stainless steel and/or nitinol and may be made using the same or different materials as the electrodes 102 and 104. The energy transfer component may be disposed in any suitable location relative to the electrodes 102, 104 so as to transfer energy to the electrodes from a power source. For example, the energy transfer component may be disposed within the electrode cage 101 or may be disposed outside the cage 101. The energy transferring device may include a coating on one or more portions, having such properties as: insulating, lubricious, therapeutic, or any other suitable properties. The energy transferring component may have a solid core or may have a hollow core and may have one or more layers.

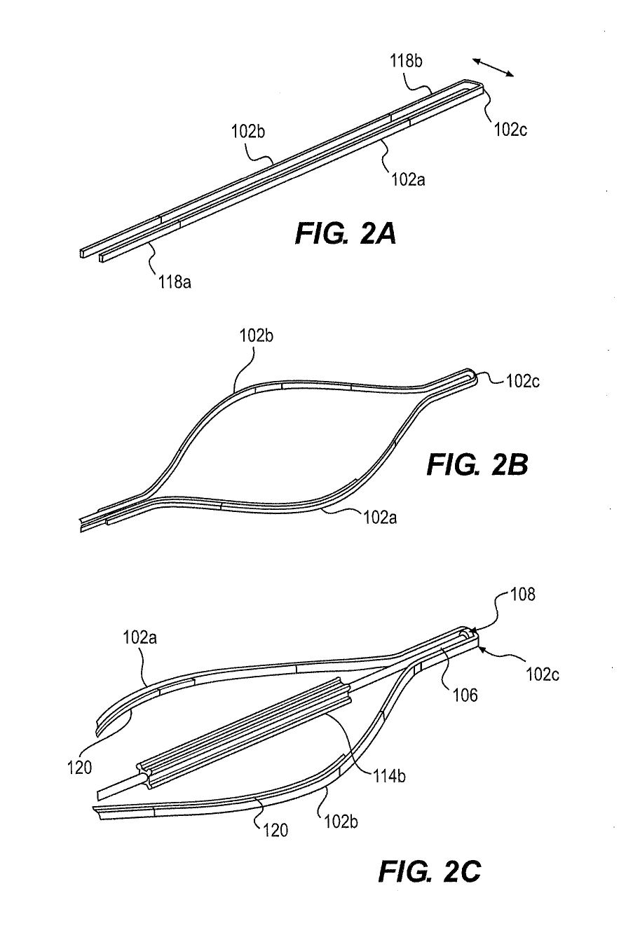

For example, the energy transfer component may be an elongate pull wire 106. The pull wire 106 may extend from a proximal portion connected to a power source, to a distal end 108 connected to one or more portions of the electrode cage 101. The distal end 108 of the pull wire 106 may have any suitable size, shape, or geometry. For example, the distal end 108 of the pull wire 106 may be have a ball or disc shape. The diameter of the pull wire 106 at the distal end may be configured to assist in attaching to the electrodes 102 and 104 or one or the other independently. For example, as shown in in FIG. 3, the distal end 108 of the pull wire 106 may have a spherical or ball shape, which may have a diameter that is different than a diameter of the adjacent portion of the pull wire 106. For example, the diameter of the distal end of the pull wire may be greater than the diameter of the adjacent portion of the pull wire 106. This increased diameter of the distal end 108 relative to the diameter of the adjacent portion of the pull wire may assist in connecting (e.g. via welding) the distal end 108 of the pull wire 106 to a portion of the electrode 102.

The distal end 108 of the pull wire 106 may be directly connected to an inner surface of the electrode 102 at a portion where the TC wire 118 and insulator 120 are not present. The energy supplied from the pull wire to the electrode 102 may be insulated from the electrode 104 having an insulating coating 118.

For example, as shown in FIGS. 2C and 3, the distal end 108 of the pull wire 106 may be adjacent to an inner surface of the bend portion 102c of the inner electrode 102 where no TC wire 120 or insulation 118 are present. The connection of the distal end 108 of the pull wire 106 and the surface of electrode 102, 104 may be made by any suitable means, such as by welding. A proximal portion of the pull wire 106 may include a coating, such as a lubricious and/or insulating coating. The pull wire 106 may also pass through the width of electrode 102 and be expanded on the opposite side to couple the distal end of the pull wire 108 to the electrode 102. The pull wire 106 may also be bonded or welded to the distal bend 102c of electrode 102 after being pulled through the electrode 102.

The device 100 may include an alignment component having one or more portions 114a and 114b that may be adapted to help maintain an angular orientation between the electrode legs. For example, an alignment component 114 may be disposed on a portion of the pull wire 106 including a portion central to cage 101. The alignment component 114 may be manufactured using any suitable material using any suitable method, such as extruding a polymer material to have an inner lumen configured to be disposed over a portion of the pull wire 106. The alignment component 114 may be assembled within the electrode cage 101 of the device

The alignment component 114 may also prevent the opposing electrodes 102 and 104 from inverting when the cage configuration 101 is expanded. For example, if the airway wall being treated restricts the expansion of the electrodes 102 and 104, buckling may occur in the inward direction causing electrode inversion. Restricting the direction of buckling may be achieved by using an alignment component 114. When the electrode 102 and 104 buckles in the inward direction, the parallel surface of the alignment component 114 may help stop inward buckling.

The alignment component 114 may have one or more portions disposed on portions of the pull wire 106, for example, alignment component 114 may have multiple portions 114a and 114b disposed along different portions of the pull wire 106. For example, as shown in in FIG. 1B, alignment component 114 may have a portion, 114a, disposed over a proximal portion of pull wire 106 and a portion of the alignment component 114b, disposed over a distal portion of the pull wire 106. Each portion, 114a and 114b, of the alignment components 114 may have any suitable size, shape, and geometry for maintaining an angular orientation between the each of the electrode legs, which may be the same or different from other portions of the alignment component 112.

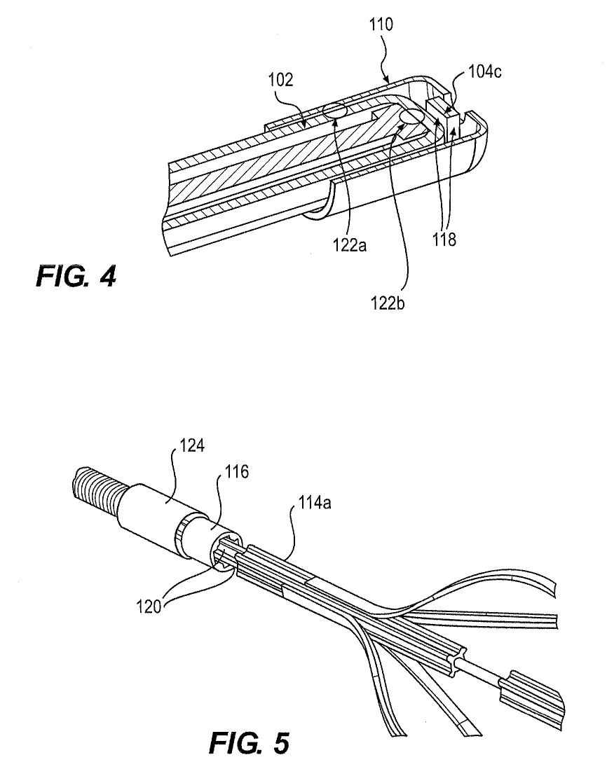

Each portion 114a and 114b of the alignment component 114 may have various features, shapes, sizes and geometries adapted to accommodate portions of the electrode legs 102a, 102b, 104a, and 104b. For example, the exterior surface of one or more portions 114 and/or 114b of the alignment component 114, may have indentations, seats, recesses, grooves, etc., to accommodate the electrode legs 102a, 102b, 104a, and 104b. For example, as shown in FIG. 5, a proximal portion 114a of the alignment component 112 may have a flat recessed features for accommodating a flat portion of an electrode leg, 102a, 102b, 104a, or 104b.

In addition to or instead of the alignment component 114, the device 100 may include an alignment insert component positioned within the distal retainer cap 110. The alignment insert portion may be manufactured using any suitable material, such as a plastic material.

The device 100 may include a distal retainer 110 disposed over a distal portion of the electrodes. For example, as shown in FIG. 1B, the distal retainer 110 may be disposed over the distal bends 102c, 104c of the electrodes 102 and 104 so that elecrode 104 may follow electrdoe 102 when the cage 101 is expanded and relaxed. The distal retainer 110 may be manufactured from any material suitable for treatment within the body and transferring energy, for example metal, such as stainless steel and/or or nitinol, and may be made using the same or different material as the electrodes 102 and 104.

The distal retainer 110 may have any suitable shape, size, and geometry. For example, as shown in FIG. 1B, the distal retainer 110 may have a cylindrical configuration having an open proximal end and a closed or partially closed distal end. The internal diameter, shape, and length of the distal retainer 110 may be configured to be disposed over the distal bends of the electrodes 102 and 104. For example, as shown in FIG. 1B, the internal cross-section of the distal retainer 110 may be adapted to be disposed over bends 102c and 104c of electrodes 102 and 104.

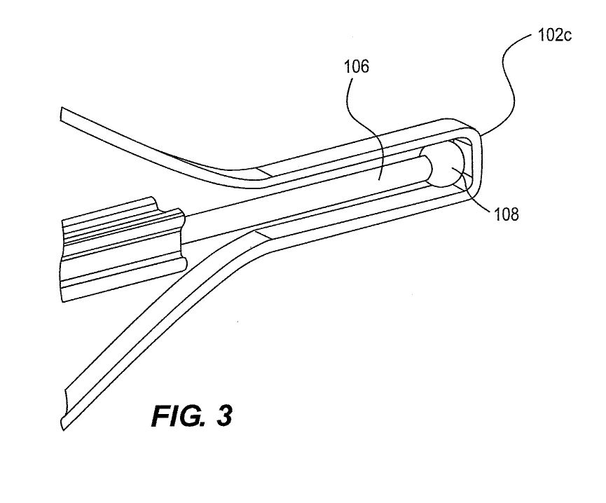

One or more of the surfaces of the distal retainer 110 may be connected to portions of the distal end of the electrodes. For example, as shown in FIG. 4, internal surfaces of the distal retainer 110 may be connected to the outside surfaces of the distal bends 102c of electrode 102 and 104c of electrode 104. The connection between the distal retainer 110 and the distal bend 102c of electrode 102 may be via any suitable methods, e.g. welding, and may be configured so as to ensure that the electrodes 102 and 104 follow each other when the device 100 is expanded and collapsed.

For example, as shown in FIG. 4, the outside surface of the bend 102c may be connected to the inside of the distal retainer 110 by any suitable means, such as via welding via a weld 122a, such as a spot weld. The inside surface bend 102c may be connected to the distal end 108 of the pull wire 106 via any suitable means, such as by welding via a weld 122b, such as a spot weld. The portions of the electrode legs 102a, 102b, 104a, and 104b having the welds 122a and 122b may have any insulating coating 118 removed.

The bend 104c on the distal end of the outer electrode pair 104 may be captivated between the bend 102c on the distal end of the inner electrode pair 102 and the distal retainer 110. This may create a bipolar relationship between electrode pair 102 and 104. Movement of the pull wire in a proximal direction may move electrode pair 102 attached to pull wire 120 via bend 102c and distal retainer cap 110 attached to an outer surface of distal bend 102c. In turn, movement of the distal retainer cap 110 disposed over bend 104c may move electrode 104 in a proximal direction. In this manner, the distal retainer 110 attached to bend 102c and disposed over bend 104c, may allow outer electrode pair 104 to follow inner electrode pair 102 attached to pull wire 120, upon actuation of the pull wire in a proximal direction via movement of the distal retainer cap in a proximal direction.

The closed distal end of the distal retainer 110 may be configured to connect to an insulator cap 112. The insulator cap 112 may be manufactured using any suitable material having insulating properties, and may have any suitable size, shape, or geometry. For example, the distal end of the distal retainer 110 may be tapered to fit inside a portion of the proximal end of the insulator cap 112.

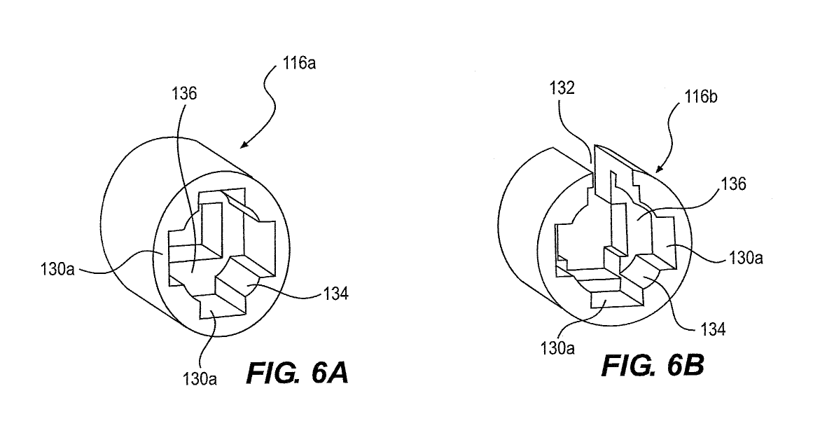

As shown in FIG. 5, the proximal ends of the electrodes 102a, 102b, 104a, and 104b may be disposed in a proximal retainer 116 such that the electrodes 102 and 104 may be retained in the radial and axial positions. The TC wires 120 also may be disposed in the proximal retainer 116. The proximal retainer 116 or a portion of the proximal retainer may be disposed inside a sleeve shrink tube 124. The proximal retainer 116 may be made using any suitable manufacturing method, for example micro molding of a polymer material. The proximal retainer 116 may be an insulator or heat and/or electrical current and may have any suitable size and shape to retain electrodes 102a, 102b, 104a, 104b, and TC wires 120. For example, as shown in FIGS. 6A and 6B, the inner lumen of the proximal retainer 116 may have a substantially circular outer cross-section. The inner lumen geometry of retainer 116 may include portions, such as straight portions 130a for retaining the electrode legs 102a, 102b, and 104a, 104b, and TC wires 120. The inner lumen geometry of the retainer 116 also may include other portions adapted to retain other components of the device 100, such as curved portions 134 for holding TC wires 120, and/or one or more stops 136 against which the proximal end of the electrode legs 102, 102b, 104a, and 104b may sit. The stops 136 may have any suitable size, shape, and geometry adapted, for example stop 136 may have a step configuration adapted to seat the proximal electrode legs 102a, 102b, 104a, and 104b.

FIG. 6A shows an example in which the proximal retainer 116a may have a closed configuration in which the electrodes 102a, 102b, 104a, 104b, and TC wires 120 may be disposed inside the proximal retainer 116a. FIG. 6B shows an example in which the proximal retainer 116b may have a slot 132 through which the TC wires 120 may be disposed and an inner portion through which the electrodes 102a, 102b, 104a, 104b, may be disposed.

The energy transfer component, such as pull wire 106, may have a distal end connected to one or more portions of the cage 101 and a proximal end connected to a power supply. The power supply may provide energy to be delivered to airway tissue via an energy transfer component, such as the pull wire 106 connected to electrodes 102 and 104. The energy supplied by the power supply may be of any suitable type, such as electrical energy (e.g. Direct Current (DC) or Alternating Current AC)), thermal energy, chemical, radiant, or any other type of energy.

For example, the power supply may supply electrical energy in the form of electrical current. The electrical current may be either AC or DC, which may be used to resistively heat the electrodes 102 and 104 via the energy transfer component, such as pull wire 106. In another example, RF energy carried by AC may also be used to inductively or resistively heat the electrodes 102 and 104 via the energy transfer component, such as pull wire 106.

The power supply may include control modes for delivering energy safely and effectively. Energy may be delivered in open loop power control mode for a specific time duration. Energy also may be delivered in temperature control mode, with output power varied to maintain a certain temperature for a specific time duration. In the case of RF energy delivery via the energy transfer component, such as pull wire 106 to the electrodes 102 and 104, the power supply may operate in an impedance control mode, in which the output power is varied to maintain a certain impedance. The embodiments of electrodes and devices described herein may be used with any suitable power supply, control algorithm (including delivery times, temperatures, etc.), handle, or any other system or device feature for energy delivery to tissue, including the system features disclosed in U.S. Pat. Nos. 7,594,925, 7,931,647, 7,740,017, 7,854,734, and 8,235,983, the complete disclosures of which are incorporated by reference herein in their entirety.

In temperature control mode, the power supply may operate at any suitable temperature, for example, up to about 75° C., or any other suitable temperature range. The duration may be varied to any suitable duration. For example, the duration may be long enough to produce the desired effect, but as short as possible to allow treatment of all of the desired target airways within a lung. For example, the duration may be 5 to 10 seconds per activation (while the device is stationary). Shorter duration with higher temperature also may produce acceptable acute effect.

Using the electrodes as described above in power control mode, power ranges of any suitable amount and any suitable duration may be used. For example, the power range may be in the range of about 10-15 W with durations of about 3-5 seconds. The power supply may include control algorithms to limit excessive thermal damage to the airway tissue by the transfer of energy from the electrodes. For example, in order to stop delivery of energy in the event of contact between airway tissue and the electrodes legs 102a, 102b, 104a, and 104b, having temperature sensing capabilities (e.g. via the TC wires 120), an algorithm may be employed to shut down energy delivery if the sensed temperature does not rise by a certain number of degrees in a pre-specified amount of time after energy delivery begins.

Another way that energy delivery may be stopped includes shutting down a power supply if the temperature is not within a predefined range at any time during energy delivery. Other algorithms include shutting down a power supply if a maximum temperature setting is exceeded or shutting down a power supply if the sensed temperature suddenly changes, such a change includes either a drop or rise, and this change may indicate failure of the temperature-sensing element.

The power supply may include various components, including a controller used to control the amount of power supplied by the power supply. The controller may include a feedback component adapted to adjust the amount of power supplied based on feedback from a feedback component, such as a sensor or any other device or component adapted to detect energy delivered to the electrodes 102 and 104, and/or the energy transfer component, such as pull wire 106.

Although the present disclosure has been described in detail with respect to devices for the treatment of airways in the lungs, it should be understood that the present disclosure also may be used for treatment of other body conduits. For example, the treatment system may be used for reducing smooth muscle and spasms of the esophagus of patients with achalasia or esophageal spasm, in coronary arteries of patients with Printzmetal's angina variant, for ureteral spasm, for urethral spasm, and irritable bowel disorders.

The devices and methods described herein provide a more effective and/or permanent treatment for asthma than those currently used.

Embodiments of the present disclosure may be used in any medical or non-medical procedure, including any medical procedure where control of air into and out of the lungs is desired. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A device for treating tissue, comprising:
a first conductive elongate member having a first distal bend portion, a first leg portion extending proximally from the first distal bend portion, and a second leg portion extending proximally from the first distal bend portion;
a second conductive elongate member having a second distal bend portion, a third leg portion extending proximally from the second distal bend portion, and a fourth leg portion extending proximally from the second distal bend portion, wherein the second distal bend portion is disposed proximally of the first distal bend portion; and
an elongate pull member affixed to the second distal bend portion.

2. The device of claim 1, wherein the first and second distal bend portions are not in the same plane.

3. The device of claim 1, wherein each of the first, second, third, and fourth leg portions form a cage configuration adapted to transition from a collapsed state to an expanded state.

4. The device of claim 1, wherein the first and second distal bend portions are not directly attached to each other.

5. The device of claim 1, further comprising an alignment element disposed on the pull member and configured to align the first and second leg portions of the first conductive elongate member and the third and fourth leg portions of the second conductive elongate member at predetermined relative orientations.

6. The device of claim 5, further comprising a proximal retainer disposed over a proximal portion of the elongate pull member, wherein the proximal retainer comprises an internal lumen having a cross-section configured to retain proximal portions of the first and second conductive elongate members and a proximal portion of the alignment element.

7. The device of claim 6, wherein the internal lumen of the proximal retainer comprises a stop configured to abut proximal ends of the first and second conductive elongate members.

8. The device of claim 1, further comprising thermocouple wires attached to one or more portions of the first and second conductive elongate members.

9. The device of claim 1, further comprising a distal retainer disposed over the first and second distal bend portions, wherein the distal retainer is directly and fixedly attached to the second conductive elongate member.

10. The device of claim, 9, wherein the distal retainer is conductive and not directly and fixedly attached to the first conductive elongate member.

11. A device for treating tissue, comprising:
a first conductive elongate member having a first distal bend portion positioned in a first plane, a first leg portion extending proximally from the first distal bend portion, and a second leg portion extending proximally from the first distal bend portion;
a second conductive elongate member having a second distal bend portion positioned in a second plane different from the first plane, a third leg portion extending proximally from the second distal bend portion, and a fourth leg portion extending proximally from the second distal bend portion, wherein the second distal bend portion is disposed proximally of the first distal bend portion,
an elongate pull member directly and fixedly attached to an inner surface of the second distal bend portion; and
an alignment element disposed on the pull member and configured to align the first and second leg portions of the first conductive elongate member and the third and fourth leg portions of the second conductive elongate member at predetermined relative orientations.

12. The device of claim 11, wherein a distal end of the elongate pull member has a diameter greater than a diameter of a proximal portion of the elongate pull member and the greater diameter distal end is directly and fixedly attached to the inner surface of the second distal bend portion.

13. The device of claim 11, wherein the first and second distal bend portions are not directly attached to each other.

14. The device of claim 11, further comprising a proximal retainer disposed over a proximal portion of the elongate pull member, wherein the proximal retainer comprises an internal lumen having a cross-section configured to retain proximal portions of the first and second conductive elongate members and a proximal portion of the alignment element.

15. The device of claim 14, wherein the internal lumen of the proximal retainer comprises a stop configured to abut proximal ends of the first and second conductive elongate members.

16. The device of claim 11, further comprising thermocouple wires attached to one or more portions of the first and second conductive elongate members.

17. The device of claim 11, further comprising a distal retainer disposed over the first and second distal bend portions, wherein the distal retainer is directly and fixedly attached to the second conductive elongate member.

18. A method of manufacturing a device for treating tissue, comprising:
bending a first elongate component to form a first set of two leg portions extending proximally of a first bent portion;
bending a second elongate member to form a second set of two leg portions extending proximally of a second bent portion; and
positioning the second bent portion approximately perpendicular to and proximal of the first bent portion.

19. The method of claim 18, further comprising attaching a distal end of a pull wire to a proximal side of the second bent portion.

20. The method of claim 18, further comprising disposing a retainer over the first and second bent portions, and directly and fixing the retainer to only one of the first and second bent portions.
